(19) United States
(12) Patent Application Publication
(10) Pub. No.: US 2017/0298392 A1
(43) Pub. Date: Oct. 19, 2017

(54) Lignin Compositions and Methods for Use in Fermentation and Animal Feed

(71) Applicant: DOMTAR PAPER COMPANY, LLC, Fort Mill, SC (US)

(72) Inventors: Mark DeANDREA, Waxhaw, NC (US); Wilton Dale MITCHELL, Rock Hill, SC (US); Neelakantam NARENDRANATH, Fort Mill, SC (US)

(21) Appl. No.: 15/486,837

(22) Filed: Apr. 13, 2017

Related U.S. Application Data
(60) Provisional application No. 62/323,238, filed on Apr. 15, 2016.

(51) Int. Cl. C12P 7/06; C12N 1/18; A23K 20/10; C12P 7/16; A23K 10/38

(52) U.S. Cl. CPC C12P 7/06; C12P 7/16; A23K 10/38; A23K 20/10; C12N 1/18

(57) ABSTRACT

This disclosure includes methods utilizing lignin or modified lignin compositions to reduce undesirable effects of unwanted microbial growth in fermentation media or animal feed, including lignin or modified lignin compositions that may be used in or for such methods.

### CROSS-REFERENCE TO RELATED APPLICATIONS

This Application claims priority to U.S. Provisional Patent Application Ser. No. 62/323,238 filed Apr. 15, 2016, which is incorporated by reference in its entirety.

### BACKGROUND

Microbial or bacterial growth or proliferation, and biofilm formation, are known to be undesirable contaminants in a variety of areas. For example, such growth, proliferation, and/or formation is typically undesirable in commercial or industrial fermentation processes. Likewise, such growth, proliferation, and/or formation is typically undesirable in animal feed such as grain and other byproducts.

Fermentation is typically performed under non-sterile conditions and can be infected or contaminated with bacteria, populations of which can reach 10⁴ to 10⁶ organisms per milliliter (organisms/ml) or more. “Fermentation” or “fermentation process” refer to a process in which a “fermentation substrate” undergoes a chemical breakdown by a “fermenting microorganism” to produce chemical or biochemical such as alcohol or organic acid. Because yeasts perform this conversion in the absence of oxygen, alcohol fermentation is considered an anaerobic process. Such fermentation processes are typically also effervescent in that they produce carbon dioxide as a byproduct. Produced alcohols can include, for example, ethanol, butanol, and the like. Such fermentation processes are well known in the art and are used, for example, to produce consumable alcohols such as beer and wine. A “fermenting microorganism” refers to bacteria, yeast, or other microorganism that will under certain conditions ferment, or perform a fermentation process on, a desired fermentation substrate to produce a desired fermentation product.

A “fermentation media” or “fermentation medium” is the material environment in which fermentation is carried out, which may include the substrate that is fermented. The fermentation substrate may, for example, be a sugar or a simple sugar such as glucose, which is metabolized by the fermenting microorganism. Fermentation substrate may also be a complex carbohydrate which can be broken down either chemically or enzymatically to simple sugars suitable for fermentation by the microorganisms. Complex carbohydrate materials suitable for fermentation include starch derived from the endosperm of the cereal grains and other seed materials, as well as, cellulose and hemicellulose in lignocellulosic materials derived from various plants. Fermentation media, including fermentation substrate and other raw materials may be processed through various means including milling, liquefaction, saccharification processes, or other desired processes either prior to or simultaneously with the fermentation process.

Bacteria that can contaminate fermentation media can, for example, belong to the lactobacillus genus, but can also include other types of bacteria such as streptococcus, bacillus, pediococcus, clostridium, and/or the like. Such contaminating bacteria may, for example, produce organic acids and other undesirable metabolites. At certain concentrations, such organic acids and/or other metabolites can hinder desirable growth of yeast, and concomitantly fermentation causing a significant reduction in productivity, yield, and/or product quality from such fermentation.

With certain feedstocks—for example wine or cider, or by-products of wine or cider—such contaminating bacteria can also degrade glycerol into acrolein, which can end up in alcohol products intended for human consumption. Such a presence of acrolein may be undesirable because studies have linked acrolein in cigarette smoke to certain cancers. Consequently, bacteriostatic and/or bactericidal methods that do not adversely affect fermentation are needed to prevent the detrimental effects caused by excessive growth of bacteria during fermentation or other processes.

### SUMMARY

In addressing the problem of bacterial contamination or infection, the present methods and compositions utilize lignin as or in an antibiotic and/or anti-microbial composition. In general, lignin is a class of complex organic polymers that form important structural materials in the support tissues of vascular plants and some algae. Chemically, lignins are cross-linked phenolic polymers. Lignin fills the spaces in the cell wall between cellulose, hemicellulose, and pectin components, especially in xylem tracheids, vessel elements, and sclereid cells. Lignin is covalently linked to hemicellulose and therefore crosslinks different plant polysaccharides, conferring mechanical strength to the cell wall and by extension the plant as a whole. As used in this disclosure, “lignin” or “lignin composition” refer to any material that is or includes lignin for the purpose of attenuating microbial growth. In some of the present embodiments, for example, the lignin can be or include kraft lignin, which is extracted from black liquor; hydrolytic lignin; lignosulfonates; organosolv lignin; soda lignin; lignin obtained by pre-treatment of lignocellulosic material; or any mixture thereof. Pre-treatment of feedstocks can include alkaline or acid pre-treatment, pre-treatment with super-critical water, and/or the like. Such lignins in a composition can be chemically, physically, and/or biologically modified. Chemical modification of lignin can include, but is not limited to, the addition of one or more organic functional groups and/or one or more inorganic functional groups. Examples of such organic functional groups include carboxyl groups, carbonyl groups, alkenyl groups, and the like. Examples of such inorganic functional groups include sodium groups, sulfate groups, potassium groups, and the like. Physical modification of lignin can include, but is not limited to extraction, milling, and/or grinding. Biological modification of lignin can be performed by biomass degradation, or incubation with microbes or enzymes.

In certain embodiments, lignin can be solubilized in ammonia such that the lignin in the present embodiments can be provided in an ammonia solution. For instance, Kraft lignin may be soluble in amounts up to 100 g dry solids per liter of ammonium hydroxide. In such embodiments, the ammonia-solubilized lignin can provide source of nitrogen in both fermentation as well as yeast propagation.

Lignin compositions can be used in fermentation processes or animal feed. Certain of the present embodiments are directed to methods or compositions to reduce undesirable effects of bacterial growth and/or bacterial metabolites. Bacterial growth in a medium can be attenuated by the inclusion of or treatment with an effective amount of lignin. The bacteria can be a Gram positive or Gram negative bacteria. More specifically, the bacteria can be a Lactobacillus, Staphylococcus, Pseudomonas, Micrococcus, Streptococcus, Klebsiella, or Escherichia. In particular examples, the bacteria can be Lactobacillus plantarum, Lactobacillus paracasei, Lactobacillus fermentum, Staphylococcus aureus, Pseudomonas aeruginosa, Micrococcus luteus, Staphylococcus epidermis, Streptococcus pyrogenes, or Escherichia coli.

In certain of the present embodiments, an effective amount of lignin can be: included or added to a solution, such as a fermentation media; or included in animal feed. “Effective amount” or “effective concentration” refers to an amount of lignin, which exhibits bacteriostatic or bactericidal properties with respect one or more contaminating bacteria, that is sufficient to inhibit or substantially prevent the growth or proliferation of the contaminating bacteria or a biofilm. An effective amount may vary based upon known factors such as pharmaceutical characteristics; the type of composition; the degree of biofilm or bacterial contamination; and the use and length of use. It is within the ability of a person of ordinary skill in the art to relatively easily determine an effective amount for lignin, such as, for example, via titration or the like. When used in connection with microbial or bacterial growth or proliferation, or biofilm formation: “inhibit” or “reduce” refer to a measurable decrease or reduction in such growth, proliferation, or formation; “prevent” refers to the complete elimination of such growth, proliferation, or formation; and “substantially prevent” refers to the elimination of at least 90% or such growth, proliferation, or formation, including, for example, a reduction in the rate of such growth, proliferation, or formation by at least 90%.

In certain of the present embodiments, the lignin is included in a growth medium, such as a fermentation medium. The addition of the lignin component can inhibit the growth of Gram positive or Gram negative bacteria. For example, the addition of a lignin component in accordance with the present embodiments can maintain bacterial concentration below 10⁴ bacteria/ml or lower, thus minimizing the effect of bacteria on fermentation product(s). A fermentation medium can include one or more of any of various known feedstocks or fermentation substrates, for example, from: forest residue; wood residue; plant roots such as sugar beet; grasses such as sugar cane; grains such as rice, corn, and/or wheat; starchy tubers such as potatoes or Jerusalem artichokes; fruit such as grapes; and/or the like. The present disclosure contemplates the use of lignin with any starch or sugar-containing material that can be fermented with yeast or other organisms to yield an alcohol or other fermentation product. The fermentation media can, but need not, include additional antibiotics such as, for example, penicillin, virginiamycin, tetracycline, and/or the like in addition to the lignin. In some embodiments, the lignin can be present at a weight percent (wt %) of a composition, solution, or fermentation medium that is substantially equal to any one of, or between any two of: 0.005. 0.01, 0.05, 0.1, 0.5, 1.0, 1.5, 2.0, 2.5, 5, 7.5, 10, 20, 30, and/or 40. The amount of lignin in certain embodiments is selected such that the growth of yeast in the fermentation medium is not substantially reduced or inhibited, i.e., such that the lignin does not substantially reduce or inhibit the growth of yeast in the fermentation medium. In certain embodiments, the fermentation media can include at least: (a) a feedstock or fermentation substrate, such as glucose or other sugar(s), at a weight percent of the fermentation media that is substantially equal to any one of, or between any two of: 1, 5, 10, 15, 20 to 25, 30, 35, 40, 45, 50, and/or 60; (b) various micronutrients, such as ammonium sulfate, K₂HPO₄, ZnSO₄, MgSO₄, and/or the like; (c) a fermenting microorganism; and (d) lignin at a weight percent of the fermentation media that is substantially equal to any one of, or between any two of: 0.005. 0.01, 0.05, 0.1, 0.5, 1.0, 1.5, 2.0, 2.5, 5, 7.5, 10, 20, 30, and/or 40. Fermenting microorganisms suitable for such fermentation processes are able to ferment or convert sugars, such as glucose or maltose, directly or indirectly into a desired fermentation product. Examples of suitable fermenting microorganisms include fungal organisms, such as yeast. One example of a yeast species suitable for at least some fermentation processes is Saccharomyces and, in particular, Saccharomyces cerevisiae.

Certain of the present embodiments are directed to methods for increasing the efficiency of alcohol production by a fermentation process. Some such methods include introducing 0.005 to 40 weight percent of a lignin to a fermentation media in a fermentation vessel, and fermenting the fermentation media to produce alcohol. In such methods, the amount of lignin is such that the lignin reduces or inhibits unwanted microbial growth in the fermentation media during alcohol production without inhibiting or substantially inhibiting the growth of yeast in the fermentation media. In some such methods, the fermentation media is maintained during fermentation at a temperature in degrees Celsius (° C.) that is substantially equal to any one of, or between any two of: 22, 24, 26, 28, 30, 32, 34, to 36, 38, and/or 40. Alternatively or additionally, in some such methods, the fermentation media is maintained during fermentation at a pH that is substantially equal to any one of, or between any two of: 2.5, 3.0, 3.5, 4.0, 4.5 to 5.0, 5.5, 6.0, 6.5, 7, 7.5, and/or 8. Certain of the present embodiments include fermentation to produce alcohol for industrial alcohol, beer, wine, and/or spirits.

In other embodiments, lignin can be used as antimicrobial during yeast propagation. “Yeast propagation” refers to an aerobic fermentation process that increases a yeast population, such as, for example, a yeast that can be used as the fermenting microorganism in a subsequent alcohol fermentation process. To limit and/or reduce the effects of such bacteria contamination, lignin can be added to a fermentation media used for yeast propagation to supplement the steam sterilization. The lignin can be added at a weight percent of the growth medium that is substantially equal to any one of, or between any two of: 0.005. 0.01, 0.05, 0.1, 0.5, 1.0, 1.5, 2.0, 2.5, 5, 7.5, 10, 20, 30, and/or 40. In at least some of the present methods, the yeast propagation process includes adding a yeast population to a growth medium that includes a carbon source and an effective amount of lignin for suppressing or inhibiting bacterial growth; incubating the growth media for a period of time under controlled conditions. Some such methods further include isolating the yeast after incubation.

Any embodiment discussed with respect to one embodiment of the invention applies to other embodiments of the invention as well and vice versa. Features described with reference to one embodiment should be understood to also be applicable to other embodiments. It is contemplated that features of any embodiment described herein can be implemented with respect to any of the present methods or compositions, and vice versa. Furthermore, the present compositions can be used in or to achieve the present methods.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5% of a non-treated reference or control.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and in the claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A shows the growth of Saccharomyces cerevisiae at 32° C. in Sabdex broth with lignin at various concentrations as measured by optical density.

FIG. 1B shows the growth of Lactobacillus plantarum at 32° C. in MRS broth with lignin at various concentrations as measured by optical density.

FIG. 1C shows the growth of Lactobacillus paracasei at 32° C. in MRS broth with lignin at various concentrations as measured by optical density.

FIG. 1D shows the growth of Lactobacillus fermentum at 32° C. in MRS broth with lignin at various concentrations as measured by optical density.

FIG. 2A shows the growth over time of Saccharomyces cerevisiae in Sabdex broth and various species of lactobacilli in MRS broth at 32° C. with no lignin.

FIG. 2B shows the growth over time of Saccharomyces cerevisiae in Sabdex broth and various species oflactobacilli in MRS broth at 32° C. with lignin at 0.05 g/mL (on dry basis).

FIG. 2C shows the growth over time of Saccharomyces cerevisiae in Sabdex broth and various species of lactobacilli in MRS broth at 32° C. with lignin at 0.1 g/mL (on dry basis).

### DETAILED DESCRIPTION OF ILLUSTRATIVE EMBODIMENTS

Embodiments of the invention relate to a lignin and its use in fermentation processes and animal feed to modulate the growth of microorganisms.

### I. FERMENTATION

Certain of the present embodiments are directed to methods of fermentation that comprise introducing an effective amount of a lignin to a fermentation medium or fermentation reaction such that the lignin will exhibit bacteriostatic or bactericidal effects in the fermentation medium, as well as lignin compositions for use in such fermentation processes. As described above, fermentation typically involves mixing fermentation media that includes: a liquid, such as water; a fermentation substrate, typically a source of sugar; and a fermenting microorganism, such as a yeast; and incubating the fermentation media under temperature and/or pH conditions that are conducive to the desired fermentation process. For example, the feedstock or fermentation substrate can be added or included at a weight percent of the fermentation media that is substantially equal to any one of, or between any two of: 1, 5, 10, 15, 20 to 25, 30, 35, 40, 45, 50, 55, and/or 60. Additionally or alternatively, the lignin can be present or added at a weight percent (wt %) of the fermentation medium that is substantially equal to any one of, or between any two of: 0.005. 0.01, 0.05, 0.1, 0.5, 1.0, 1.5, 2.0, 2.5, 5, 7.5, 10, 20, 30, and/or 40. In some embodiments, the fermentation media can also include various micronutrients, such as ammonium sulfate, K2HPO4, ZnSO4, MgSO4, and/or the like. Examples of suitable fermenting microorganisms include fungal organisms, for example a yeast such as Saccharomyces or Saccharomyces cerevisiae. In some such methods, the fermentation media is maintained during fermentation at a temperature in degrees Celsius (° C.) that is substantially equal to any one of, or between any two of: 22, 24, 26, 28, 30, 32, 34, to 36, 38, and/or 40. Alternatively or additionally, in some such methods, the fermentation media is maintained during fermentation at a pH that is substantially equal to any one of, or between any two of: 2.5, 3.0, 3.5, 4.0, 4.5 to 5.0, 5.5, 6.0, 6.5, 7, 7.5, and/or 8.

Fermentation is used in many industries for a variety of applications, including for example the production of alcoholic beverages, bread baking, and the production of ethanol. For example, the use of ethanol as a gasoline additive not only reduces the emission of harmful air pollutants, but can also lower dependence on imported fossil fuels.

In one example of an industrial fermentation process, ethanol can be produced in dry-grind corn milling plants. Dry-grind corn milling involves milling, cooking, enzyme addition, and fermentation by yeasts in water. During fermentation in a corn dry milling facility, only 30-35% of the corn is actually converted to ethanol, about one-third to CO₂, and one-third remains as dissolved organics and suspended solids in the (whole) stillage after ethanol removal by distillation in distillation columns. The stillage typically contains about 89% water.

Other carbohydrate feedstocks or fermentation substrates are also known for use as a fermentation substrate for producing ethanol in yeast fermentation processes. Such feedstocks vary by region, typically according to price and availability in a given region. For example, the dominant ethanol feedstock in warmer regions is sugarcane. In temperate regions, corn or sugar beets are used. In the U.S., the main feedstock for the production of ethanol is currently corn. Approximately 10.6 liters of ethanol are produced from one bushel of corn, approximately 0.42 liter per kilogram. Although most of the fermentation plants in the U.S. have been built in corn-producing regions, sorghum is also an important feedstock for ethanol production in the Plains states. Pearl millet is showing promise as an ethanol feedstock for the southeastern U.S. and the potential of duckweed is being studied. In some parts of Europe, particularly France and Italy, grapes have become a common feedstock for fuel ethanol by the distillation of surplus wine. In Japan, it has been proposed to use rice as an ethanol source. In addition, forestry-based biomass such as wood, wood chips, forestry residue, and the like can also be used. Forestry/wood based biomass can contain a major proportion of lignocellulosic matter are suitable candidates for conversion to liquid and gaseous fuels.

One example of an alcoholic fermentation process can include Saccharomyces cerevisiae, also known as baker's yeast, incubated at an appropriate temperature, such as 30° C., in a fermentation medium, such as water, that also contains a fermentation substrate, such as glucose at a weight percent of 1 to 20 wt. %, and various micronutrients, such as ammonium sulfate, K₂HPO₄, ZnSO₄, and MgSO₄ at a weight percent of 0.0001 to 0.5% per micronutrient.

In the present methods, an effective amount of lignin component or lignin composition is also included at a weight percent of the fermentation media between 0.005 and 40.

The fermentation medium can then be placed in a fermentation vessel and inoculated with a yeast. The vessel is typically covered to avoid the evaporative escape of ethanol produced by the fermentation process. The vessel is typically incubated at about 30° C. for a specified period of time, for example 48 hours, and is sometimes stirred or shaken. Samples can be drawn and sample content analyzed prior to, during, and/or after fermentation. Calculation of ethanol content in the samples can be calculated using the following formula: Ethanol (mg/g)=(Sample peak area)/(Standard peak area)×(Concentration of standard)×(Dilution of sample). The ethanol content can be expressed in % grams of ethanol produced per 100 g of fermentation media.

### II. ANTIMICROBIAL ADDITIVES TO ANIMAL FEED

In some of the present embodiments, an effective amount of lignin can also be added to or included in animal feed, such as at a weight percent of the animal feed substantially equal to any one of, or between any two of: 0.005. 0.01, 0.05, 0.1, 0.5, 1.0, 1.5, 2.0, 2.5, 5.0, 7.5, 10.0, 12.5, 15.0, 17.5, and/or 20.

For example, in the example of a traditional dry-grind ethanol production process summarized above, more than 75% of the solids in stillage are removed by centrifugation. This solids fraction, known as thick stillage is dried to a product known as distillers dried grains (DDG). The excess liquid centrate resulting from the centrifugation, known as thin stillage, is evaporated to produce syrup, which is usually added to the centrifuged solids prior to drying. The dried product from this combination is known as DDG with solubles (DDGS), and is often sold as animal feed. DDGS are low in essential amino acids, particularly lysine (about 0.7%), and methionine (about 0.3%). This limits the use of DDGS in animal feed primarily to ruminant animals such as cows and sheep. As a result, DDGS are largely unsuitable as feed for monogastric animals.

Because the present fermentation methods include the addition of an effective amount of lignin, DDGS resulting from such fermentation methods may already include an effective amount of lignin. Additionally, because lignin is already on-site for use in such methods, further lignin may be practically and economically added to the DDGS resulting from such methods. Additional lignin can also supply additional antioxidant properties or capacity to the animal feed.

### III. YEAST PROPAGATION

Yeast propagation typically includes adding a yeast population to an appropriate growth medium that includes a carbon source, and the growth medium including the yeast population is incubated under appropriate propagation conditions for a period of time, for example 48 hours. After incubation, the yeast may be isolated from the growth media for subsequent use, such as in an alcohol fermentation process. Similar to the contamination discussed above for alcohol fermentation, during propagation of yeast, such as Saccharomyces cerevisiae, lactic acid bacterial contamination can occur. Traditional antibiotics typically cannot be used during the yeast propagation process and, as a result, stringent steam sterilization of equipment and vessels is typically used. Nevertheless, such steam sterilization is not always entirely effective and bacteria sometimes still contaminate the yeast propagation process. To limit and/or reduce the effects of such bacteria contamination, lignin can be added to a growth medium to supplement the steam sterilization. The lignin can be added, for example, in concentrations similar to those discussed above for alcohol fermentation, specifically, at a weight percent of the growth medium that is substantially equal to any one of, or between any two of: 0.005. 0.01, 0.05, 0.1, 0.5, 1.0, 1.5, 2.0, 2.5, 5, 7.5, 10, 20, 30, and/or 40. In at least some of the present methods, the yeast propagation process includes adding a yeast population to an appropriate growth medium that includes a carbon source and an effective amount of lignin for suppressing or inhibiting bacterial growth; incubating the growth media for a period of time, such as 48 hours or more, and under controlled conditions, such as temperature and/or pH. In some such methods, the growth medium is maintained during incubation at a temperature in degrees Celsius (° C.) that is substantially equal to any one of, or between any two of: 28, 30, 32, 34, to 36, 38, and/or 40. Alternatively or additionally, in some such methods, the growth medium is maintained during incubation at a pH that is substantially equal to any one of, or between any two of: 2.5, 3.0, 3.5, 4.0, 4.5 to 5.0, 5.5, 6.0, 6.5, 7, 7.5, and/or 8. Some such methods further include isolating the yeast after incubation.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Lactic acid bacterial species such as Lactobacillus plantarum (ATCC14917), L. paracasei (ATCC25598), and L. fermentum (ATCC14931) were obtained from the American Type Culture Collection. The yeast, Saccharomyces cerevisiae (C6 Fuel) was obtained from Lallemand Biofuels & Distilled Spirits. These strains were used as test organisms for evaluating antimicrobial effects of lignin. The chosen lactic bacterial strains are known potential contaminants in fermentation production of ethanol. The production organism, S. cerevisiae, was also included in the study to determine whether the level of lignin required to control the bacteria would also have a detrimental effect on yeast.

The bacterial strains and the yeast were cultured overnight in MRS broth and Sabdex broth, respectively, for approximately 21 hours (h) at 32° C.

Lignin from the Kraft pulping process obtained from Domtar Mill at Plymouth, N.C. was added into various test tubes with 8.5 milliliters (mL) MRS broth or 10 mL Sabdex broth to final concentrations of 0.001, 0.01, 0.025, 0.05, 0.075, 0.1, and 0.125 g/mL (on a dry basis). One test tube each for MRS and Sabdex broth was used with no lignin to serve as control for lactic bacteria and yeast, respectively. Each test tube was inoculated with 150 μL of each of the overnight cultures of bacteria and yeast to a final concentration of approximately 10⁶ colony forming units per milliliter (CFU/mL) of bacteria and approximately 10⁵ CFU/mL of the yeast.

The inoculated test tubes were then incubated at 32° C., and one (1) mL of the inoculated media in the tubes was sampled at each of 0 h, 24 h, and 48 h. The samples were then diluted 10 fold and used for optical density measurement at 600 nm using a UV-1800 Shimadzu UV-VIS spectrophotometer. A set of test tubes with just the respective media and various amounts of lignin without bacterial or yeast inoculation was used to serve as the corresponding blanks for optical density measurements.

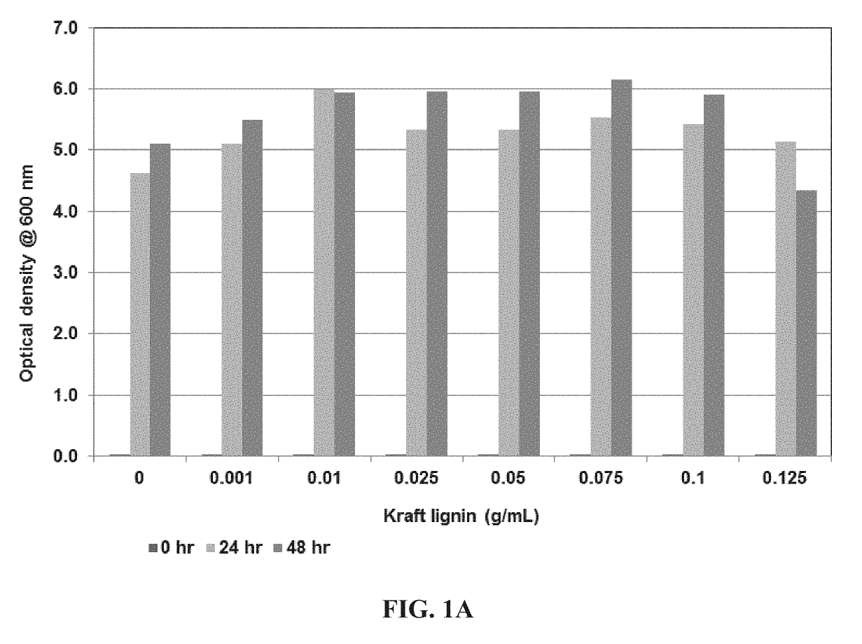

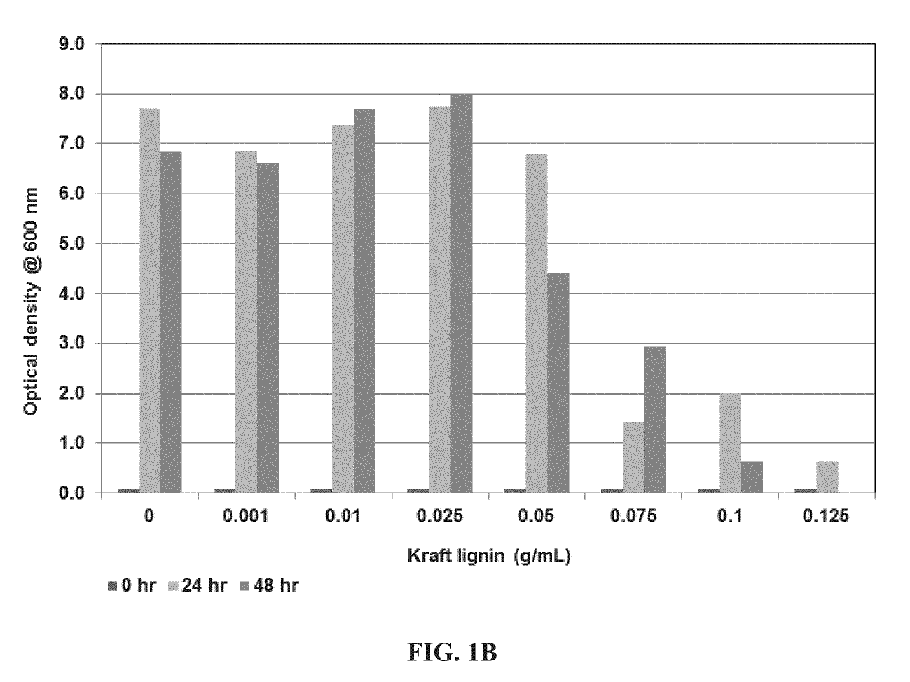

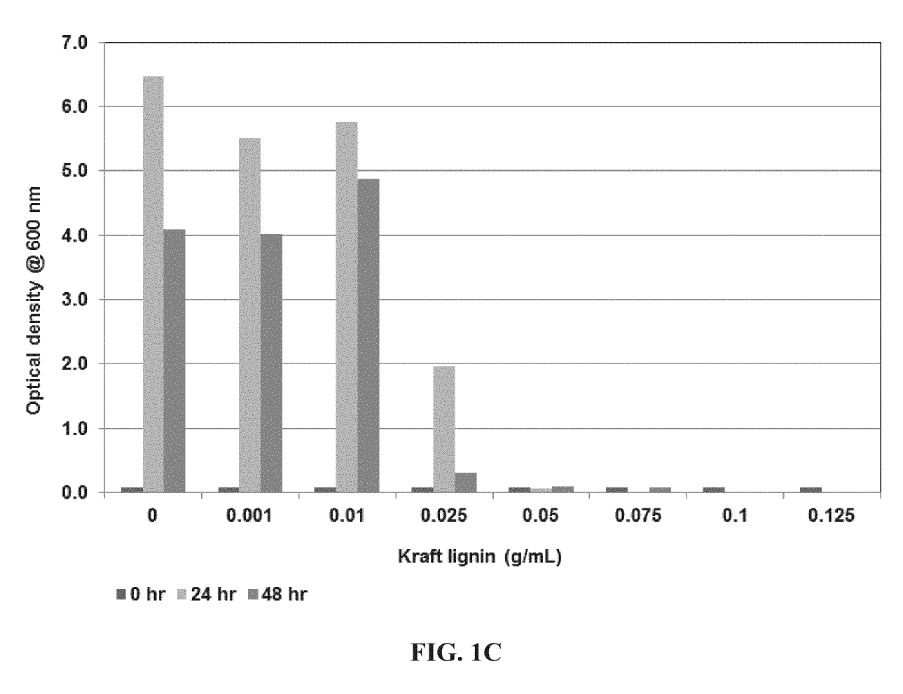

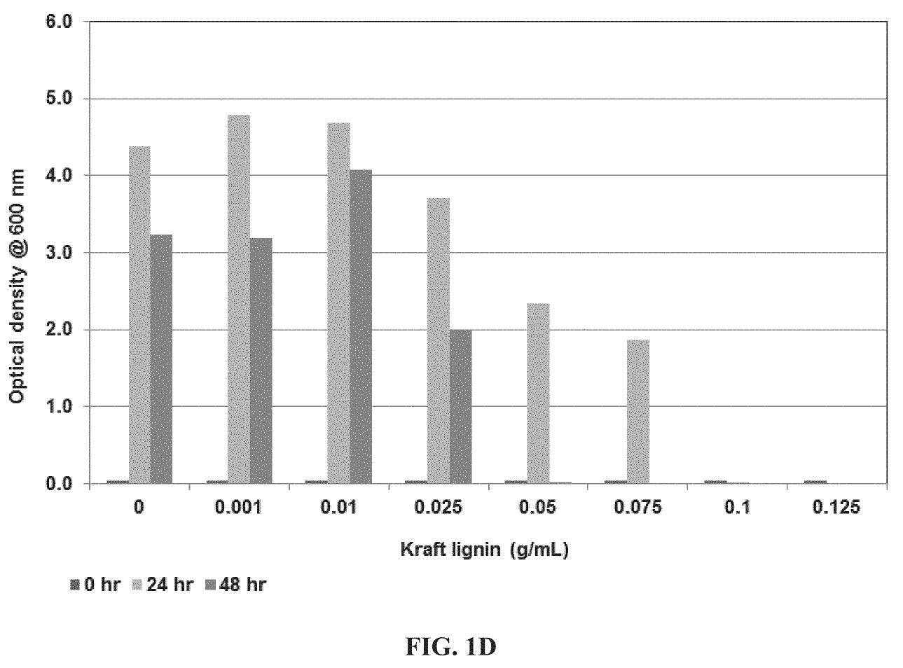

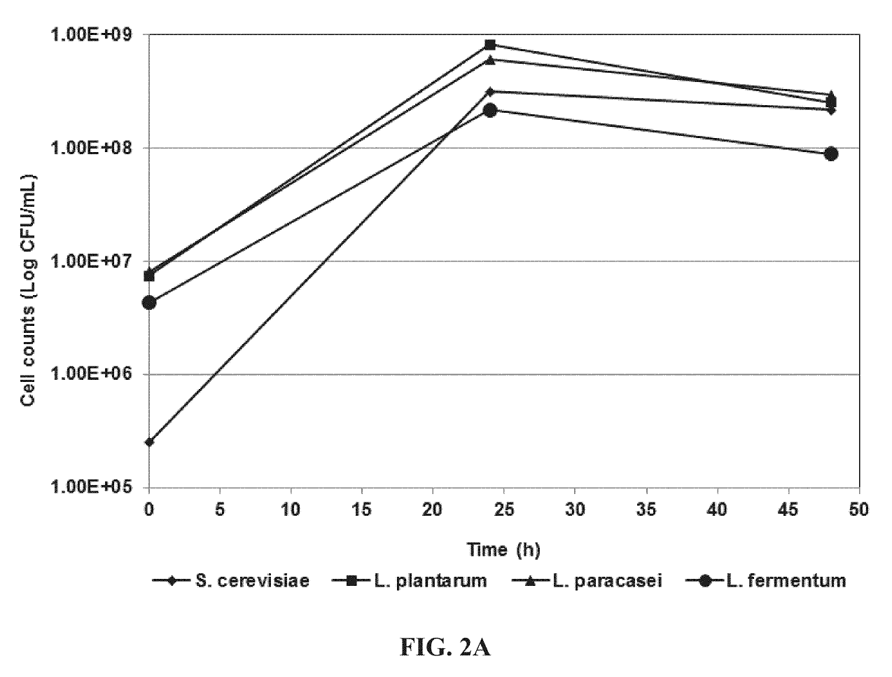

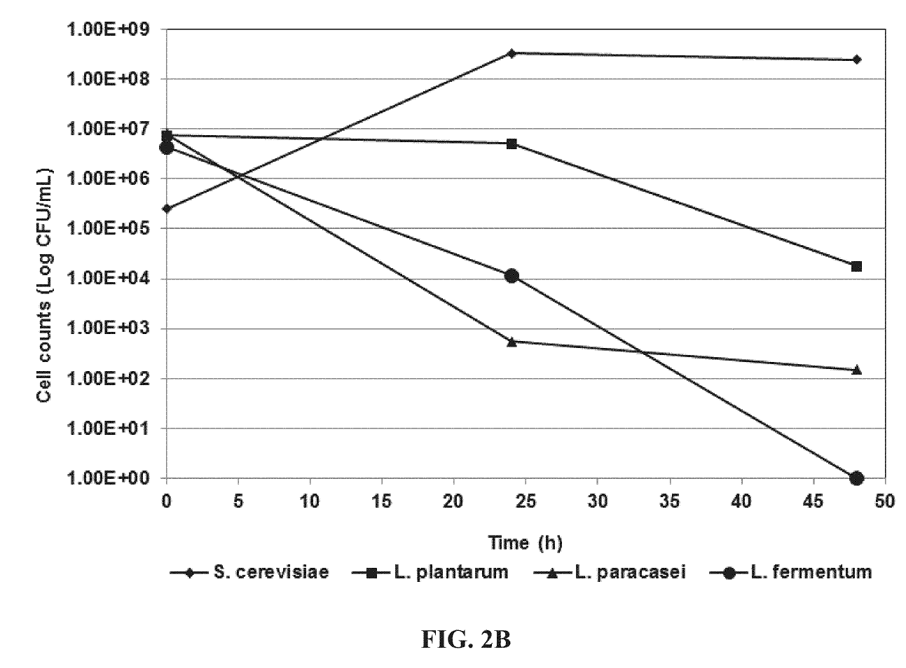

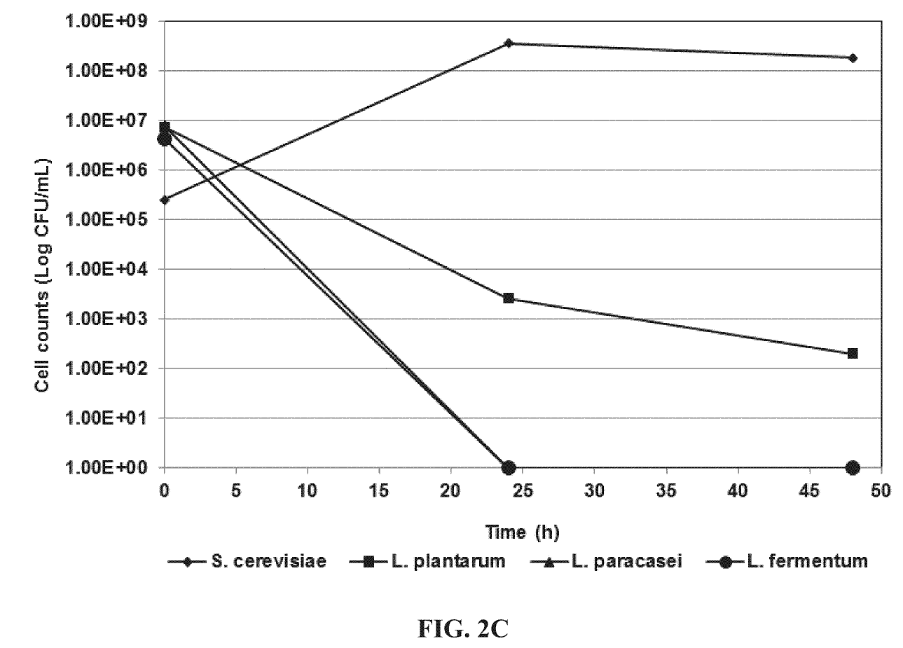

An additional one (1) mL of the inoculated media from each of the test tubes with no lignin, 0.05, and 0.1 g/mL lignin was sampled at each of 0 h, 24 h, and 48 h. These additional samples were serially diluted and plated on to MRS agar plates, which plates were then incubated at 32° C. for 48-72 hours before colonies were enumerated. The results are shown in FIGS. 1A-1D and FIGS. 2A-2C.

In brief, FIG. 1A shows the growth of Saccharomyces cerevisiae at 32° C. in Sabdex broth with lignin at various concentrations as measured by optical density, demonstrating that lignin does not adversely alter Saccharomyces cerevisiae growth. FIG. 1B shows the growth inhibition of Lactobacillus plantarum at 32° C. in MRS broth with lignin at various concentrations as measured by optical density, demonstrating the antibacterial effect of lignin in the bacterial growth medium. FIG. 1C shows the growth inhibition of Lactobacillus paracasei at 32° C. in MRS broth with lignin at various concentrations as measured by optical density, demonstrating the antibacterial effect of lignin in the bacterial growth medium. FIG. 1D shows the growth inhibition of Lactobacillus fermentum at 32° C. in MRS broth with lignin at various concentrations as measured by optical density, demonstrating the antibacterial effect of lignin in the bacterial growth medium.

FIG. 2A shows the growth over time of Saccharomyces cerevisiae in Sabdex broth and various species of lactobacilli in MRS broth at 32° C. with no lignin, providing a base line for comparison and demonstration of lignin effectiveness. FIG. 2B shows the growth over time of Saccharomyces cerevisiae in Sabdex broth and various species oflactobacilli in MRS broth at 32° C. with lignin at 0.05 g/mL (on dry basis), demonstrating inhibitory effects on lactobacilli and not Saccharomyces cerevisiae. FIG. 2C shows the growth over time of Saccharomyces cerevisiae in Sabdex broth and various species of lactobacilli in MRS broth at 32° C. with lignin at 0.1 g/mL (on dry basis), with the increased concentration of lignin resulting in greater inhibition of lactobacilli and minimal if any inhibition of Saccharomyces cerevisiae.

As can be seen from the test results, at least 0.05 g/mL (5 weight percent of lignin) is able to substantially inhibit the lactobacilli tested while yeast, the production organism is minimally inhibited, if at all, even at the highest concentration of lignin tested, 0.125 g/mL (12.5 weight percent). This makes lignin one of the choices for use as a natural (non-antibiotic) antimicrobial in the ethanol production process or any yeast based fermentation processes where bacterial contaminants are an issue.

The above specification and examples provide a complete description of the methods and compositions of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A method for reducing microbial growth in an alcohol fermentation medium, the method comprising:
adding an effective amount of a lignin or modified lignin to the fermentation medium to inhibit growth of at least one microbe in the alcohol fermentation medium.

2. The method of claim 1, wherein the alcohol production is ethanol or butanol production.

3. The method of claim 1, wherein the at least one microbe includes a bacteria.

4. The method of claim 3, where the bacteria is Gram positive.

5. The method of claim 3, wherein the bacteria is a Lactobacillus, Staphylococcus, Pseudomonas, Micrococcus, Streptococcus, Klebsiella, or Escherichia.

6. The method of claim 1, where the lignin is added at a weight percent of the fermentation media of 0.005 to 20, the fermentation media is disposed in a fermentation vessel, and the method further comprises:
fermenting the fermentation media to produce alcohol;
where the fermentation media is maintained during fermenting at a temperature of between 30 to 38° C. and a pH of between 2.5 to 6, and wherein the lignin or modified lignin reduces or inhibits growth of at least one microbe in the fermentation media during fermenting without inhibiting the growth of yeast in the fermentation media.

7. The method of claim 6, wherein the at least one microbe includes a bacteria.

8. The method of claim 6, wherein the alcohol produced is ethanol or butanol.

9. The method of claim 8, where the lignin is added at a weight percent of the fermentation media of 2.5 to 7.5.

10. The method of claim 8, where the bacteria is Gram positive.

11. The method of claim 8, wherein the bacteria is a Lactobacillus, Staphylococcus, Pseudomonas, Micrococcus, Streptococcus, Klebsiella, or Escherichia.

12. A fermentation medium comprising lignin or modified lignin in an amount of 0.005 to 40 weight percent of the fermentation medium.

13. The fermentation medium of claim 12, wherein the lignin comprises one or more lignins selected from the group consisting of: kraft lignin, hydrolytic lignin, lignosulfonates, organosolv lignin, soda lignin, and any mixture thereof.

14. The fermentation medium of claim 12, where the lignin or modified lignin comprises 0.005 to 20 weight percent of the fermentation medium.

15. The fermentation medium of claim 14, wherein the fermentation medium is an alcohol fermentation medium.

16. The fermentation medium of claim 12, further comprising a Gram positive bacteria the growth of which is inhibited by the lignin or modified lignin.

17. Animal feed comprising:
distillers dried grains (DDG); and
lignin or modified lignin;
where the lignin is present in a weight percent of 0.05 to 5 percent of the animal feed.

18. The animal feed of claim 17, where the DDG comprises DDG with solubles (DDGS).

19. A method for propagating yeast, the method comprising:
adding an effective amount of a lignin or modified lignin to a propagation medium to inhibit growth of at least one microbe in the propagation medium.

20. The method of claim 19, wherein the lignin is 0.005 to 40 weight percent of the propagation medium.
